SEQUENCE LISTING (32 sequences)

SEQ ID NO: 1 - DNA, 20 bp, Artificial Sequence
  forward primer
tatcaagcaacctgcctcaa

SEQ ID NO: 2 - DNA, 23 bp, Artificial Sequence
  backward primer
tggcataaaacaatgtgaagaga

SEQ ID NO: 3 - DNA, 18 bp, Artificial Sequence
  forward primer
cccagtgggcagtctgaa

SEQ ID NO: 4 - DNA, 20 bp, Artificial Sequence
  backward primer
cttgcatttggtggatgcta

SEQ ID NO: 5 - DNA, 20 bp, Artificial Sequence
  primer sequence for CCR1
gtggtgggcaatgtcctagt

SEQ ID NO: 6 - DNA, 20 bp, Artificial Sequence
  primer sequence for CCR1
tcagattgtagggggtccag

SEQ ID NO: 7 - DNA, 23 bp, Artificial Sequence
  primer sequence for CCR2
gtatccaagagcttgatgaaggg

SEQ ID NO: 8 - DNA, 26 bp, Artificial Sequence
  primer sequence for CCR2
gtgtaatggtgatcatcttgtttgga

SEQ ID NO: 9 - DNA, 20 bp, Artificial Sequence
  primer sequence for CCR3
gcaccaccctgtgaaaaagt

SEQ ID NO: 10 - DNA, 20 bp, Artificial Sequence
  primer sequence for CCR3
cgaggactgcaggaaaactc

SEQ ID NO: 11 - DNA, 20 bp, Artificial Sequence
  primer sequence for CCR4
aggcaaggaccctgacctat

SEQ ID NO: 12 - DNA, 20 bp, Artificial Sequence
  primer sequence for CCR4
ggactgcgtgtaagaggagc

SEQ ID NO: 13 - DNA, 20 bp, Artificial Sequence
  primer sequence for CCR5
attctccacaccctgtttcg

SEQ ID NO: 14 - DNA, 20 bp, Artificial Sequence
  primer sequence for CCR5
tcaggcttgtcttgctggaa

SEQ ID NO: 15 - DNA, 20 bp, Artificial Sequence
  primer sequence for CCR6
gtggtgatgacctttgcctt

SEQ ID NO: 16 - DNA, 20 bp, Artificial Sequence
  primer sequence for CCR6
aggaggaccatgttgtgagg

SEQ ID NO: 17 - DNA, 20 bp, Artificial Sequence
  primer sequence for CCR7
aacgggctggtgatactgac

SEQ ID NO: 18 - DNA, 20 bp, Artificial Sequence
  primer sequence for CCR7
atgaagactaccaccacggc

SEQ ID NO: 19 - DNA, 20 bp, Artificial Sequence
  primer sequence for CCR8
ttcctgcctcgatggattac

SEQ ID NO: 20 - DNA, 20 bp, Artificial Sequence
  primer sequence for CCR8
gcttccacctcaaagactgc

SEQ ID NO: 21 - DNA, 20 bp, Artificial Sequence
  primer sequence for D6
tcttcatcacctgcatgagc

SEQ ID NO: 22 - DNA, 20 bp, Artificial Sequence
  primer sequence for D6
tatgggaaccacagcatgaa

SEQ ID NO: 23 - DNA, 20 bp, Artificial Sequence
  primer sequence for CCR12
ctggcggtgtttatcttggt

SEQ ID NO: 24 - DNA, 20 bp, Artificial Sequence
  primer sequence for CCR12
aaccagcagaggaaaagcaa

SEQ ID NO: 25 - DNA, 24 bp, Artificial Sequence
  primer sequence for GAPDH
catcctgcaccaccaactgcttag

SEQ ID NO: 26 - DNA, 24 bp, Artificial Sequence
  primer sequence for GAPDH
gcctgcttcaccaccttcttgatg

SEQ ID NO: 27 - DNA, 19 bp, Artificial Sequence
  forward primer for MCP-1
gtctctgtcacgcttctgg

SEQ ID NO: 28 - DNA, 20 bp, Artificial Sequence
  reverse primer for MCP-1
gatctctctcttgagcttgg

SEQ ID NO: 29 - DNA, 20 bp, Artificial Sequence
  forward primer for CCR11
ctggcggtgtttatcttggt

SEQ ID NO: 30 - DNA, 20 bp, Artificial Sequence
  reverse primer for CCR11
aaccagcagaggaaaagcaa

SEQ ID NO: 31 - DNA, 1035 bp, Homo sapiens
  misc_feature (1)..(1035): hCCR12 chemokine receptor sequence
atggccaattacacgctggcaccagaggatgaatatgatgtcctcatagaaggtgaactggagagcgatgaggcagagcaatgtgacaagtatgacgcccaggcactctcagcccagctggtgccatcactctgctctgctgtgtttgtgatcggtgtcctggacaatctcctggttgtgcttatcctggtaaaatataaaggactcaaacgcgtggaaaatatctatcttctaaacttggcagtttctaacttgtgtttcttgcttaccctgcccttctgggctcatgctgggggcgatcccatgtgtaaaattctcattggactgtacttcgtgggcctgtacagtgagacatttttcaattgccttctgactgtgcaaaggtacctagtgtttttgcacaagggcaactttttctcagccaggaggagggtgccctgtggcatcattacaagtgtcctggcatgggtaacagccattctggccactttgcctgaattcgtggtttataaacctcagatggaagaccagaaatacaagtgtgcatttagcagaactcccttcctgccagctgatgagacattctggaagcattttctgactttaaaaatgaacatttcggttcttgtcctccccctatttatttttacatttctctatgtgcaaatgagaaaaacactaaggttcagggagcagaggtatagccttttcaagcttgtttttgccgtaatggtagtcttccttctgatgtgggcgccctacaatattgcatttttcctgtccactttcaaagaacacttctccctgagtgactgcaagagcagctacaatctggacaaaagtgttcacatcactaaactcatcgccaccacccactgctgcatcaaccctctcctgtatgcgtttcttgatgggacatttagcaaatacctctgccgctgtttccatctgcgtagtaacaccccacttcaacccagggggcagtctgcacaaggcacatcgagggaagaacctgaccattccaccgaagtgtaa

SEQ ID NO: 32 - DNA, 1083 bp, Mus sp.
  misc_feature (1)..(1083): mCCR12 chemokine receptor sequence
atggataactacacagtggccccggacgatgaatatgatgtcctaatcttagacgactacctggacaacagtgggccggaccaagttccggcccccgagttcctctccccccagcaggtgctgcagttctgctgcgcggtgtttgcggtgggtctcttggacaacgtgctggcggtgtttatcttggtgaaatacaaaggactcaagaatctggggaacatctacttcctaaacctggcactttcaaacctgtgtttcctgcttcccctgccgttctgggcccatactgcagcacacggggaaagccctggcaacgggacctgtaaagttcttgtcggactccactcctcgggcttatacagcgaggtgttttccaacatcctcctccttgtgcaaggatacagggtgttttcccaagggcgactggcctccatcttcacgacagtgtcttgtggtattgttgcgtgcatcctggcatgggccatggctactgcgctctctttgcccgagtctgtgttttatgagcctcggatggaaagacagaaacacaagtgtgcctttggcaaacctcacttcttgccaatcgaagcgccgctctggaagtacgttctgacgtcaaaaatgatcatcttggtacttgcttttcctctgctggtttttataatctgctgcaggcaactgaggagaaggcagagcttcagggagagacagtacgacctccacaagccggctcttgtcataacgggcgtgttccttttgatgtgggcgccttacaacactgtgcttttcctgtctgctttccaggaacacttgtccctgcaggatgagaagagcagctaccacctggacgcaagtgttcaggtcacacagctggtagcgaccacccactgctgcgtcaacccgctgctctatttgcttcttgaccggaaggcctttatgagataccttcgcagcctgttcccacggtgcaatgatatcccctatcaaagtagtggaggctatcagcaagcgcctccaagggaaggtcatggcaggcccattgaactgtacagcaatttgcatcaaaggcaggatataatataa

(19) United States
(12) Patent Application Publication
(10) Pub. No.: US 2004/0096913 A1
(43) Pub. Date: May 20, 2004

(54) Cloning and expression of a new MCP receptor in glial cells

(76) Inventors: Erik Hendrikus, G., M. Boddeke, Haren (NL); Knut Peter, H. Biber, Mensingeweer (NL)

Correspondence Address: TRASK BRITT, P.O. BOX 2550, SALT LAKE CITY, UT, 84110, US

(21) Appl. No.: 10/623,472

(22) Filed: Jul. 18, 2003

Related U.S. Application Data
(63) Continuation of application No. PCT/NL02/00039, filed on Jan. 18, 2002.

(30) Foreign Application Priority Data
Jan. 18, 2001 (EP) 01200181.4

(51) Int. Cl. C12Q 1/68; G01N 33/53; G01N 33/567

(52) U.S. Cl. 435/7.2; 435/6

(57) ABSTRACT

The invention relates to the fields of inflammation and immunology and, more specifically, to the field of chemokines and receptors therefor, and their role in neurodegenerative or neuroinflammatory disease. The invention provides a method for identifying a candidate drug compound for the treatment of inflammatory or degenerative brain disease comprising testing the compound for its capacity to modulate or mimic MCP-1 binding with a chemokine receptor capable of being expressed on brain glial cells, the receptor known in the mouse as L-CCR or in humans as CRAM-B.

### CROSS-REFERENCE TO RELATED APPLICATION

This application is a continuation of PCT International Patent Application No. PCT/NL/02/00039, filed on Jan. 18, 2002, designating the United States of America, and published, in English, as PCT International Publication No. WO 02/057779 on Jul. 25, 2002, the contents of the entirety of which is incorporated by this reference.

### TECHNICAL FIELD

The invention relates to the fields of inflammation and immunology and, more specifically, to the field of chemokines and receptors therefor, and their role in neurodegenerative or neuroinflammatory disease.

### BACKGROUND

Chemokines are small chemotactic cytokines of approximately 10 kDa, which orchestrate the inflammatory response by attracting leukocytes to sites of inflammation and by controlling the homing of dendritic cells, T-cells and B-cells (for review see: 1, 2, and 3). Chemokines and their receptors, all of which are G-protein coupled, are subdivided into four families: CXC-, CC-, C- and CX3C-chemokines (3). Chemokine signaling is highly promiscuous, most chemokines activating more than one chemokine receptor and vice versa (4, 5). In humans, more than 25 CC chemokines and 10 CC chemokine receptors (CCR) have been cloned (3). Furthermore, it is likely that some of the currently known orphan chemokine receptors will make chemokine signaling even more complex (4).

Ishizuka et al., in “Identification of Monocyte Chemoattractant Protein-i in Senile Plaques and Reactive Microglia of Alzheimer's Disease” (Psych. Clin. Neurosci. 1997), stress the importance of the chemokine MCP-1 in the pathology of Alzheimer's disease. However, no mention of involvement of specific chemokine receptors has been made. Also, Berman et al., in “Localization of Monocyte Chemoattractant Peptide-1 Expression in the Central Nervous System in Experimental Autoimmune Encephalomyelitis and Trauma in the Rat” (J. Immunol. 156, 1996), shows expression of the chemokine MCP-1 in experimental autoimmune encephalitis (EAE), a model for MS. From experiments using MCP-1 knock-out mice, the importance of MCP-1 in EAE pathology is now well established. The MCP-1 receptor currently considered important by Bermann et al. is CCR2. The publication thus focuses on MCP-1 and CCR2 knock-out mice. Koch et al., in “Enhanced Production of Monocyte Chemoattractant Protein-1 in Rheumatoid Arthritis” (J. Clin. Invest. 1992), again clearly supports a role of the chemokine MCP-1 in rheumatoid arthritis and the subsequent chemoattraction of synovial tissue macrophages, but does not mention involvement of chemokine receptors at all. PCT International Publications WO 00/46195 and WO 00/46197 concern anti-inflammatory indole derivatives which interfere with diseases mediated by the chemokines MCP-1 and RANTES. It is suggested that the indole derivatives act as inhibitors of the MCP-1 receptor CCR2. PCT International Publication WO 00/69815 suggests the use of ureido-substituted cyclic amine derivatives as inhibitors of the chemokines MCP-1 and MIP-1α involved in a variety of diseases.

Chemokines and their receptors are not only found in the peripheral immune system. It has recently become clear that chemokines are also expressed in the brain during development and brain pathology (for review, see also, 6, 7, 8, & 9). One of the first described and most prominent chemokines in the brain is monocyte chemoattractant protein-1 (MCP), which is found in brain tissue after ischemia (10, 11), Alzheimer's disease (12), and Multiple Sclerosis (13, 14 15). Within the damaged brain, MCP-1 is produced by both astrocytes and microglia (10) and mediates, presumably, the infiltration by monocytes/macrophages and lymphocytes (16, 17). Both astrocytes and microglia are not only capable to produce chemokines but they also are involved in chemokine signaling since it is known that glial cells express functional chemokine receptors (18, 19, 20). In cultured microglia and astrocytes, MCP-1 induces transient increases in intracellular Ca²⁺ and/or chemotaxis (20, 21, 22, 23, 24). Although cultured glial cells (astrocytes and microglia) respond to MCP-1 stimulation, possible mRNA expression of the corresponding chemokine receptor for MCP-1 (CCR 2) (25, 26) is controversial in glial cells (18, 24, 9), and so far, CCR2 mRNA expression has not been found in astrocytes.

### BRIEF SUMMARY OF THE INVENTION

The invention provides the insight that, possibly instead of activating the CCR-2 receptor, in brain cells, such as glial cells, MCP-1 activates at least one other CC chemokine receptor, a receptor earlier known as orphan receptor L-CCR in the mouse or CRAM-B in humans (hereafter “CCR12” or “CCR11”), depending on final classification by the committee on nomenclature, see also FIGS. 8, 8A, 8B. With this insight, the invention provides a method for obtaining or identifying an agonist or antagonist of degenerative or inflammatory disease comprising testing a candidate agonist or antagonist compound in a method according to one as provided herein and determining the compound's capacity to modulate or mimic MCP-1 binding to the receptor in the method. After synthesizing the thus-identified compound in desirable quantities, the invention thus allows for the production of an agonist or antagonist of degenerative or inflammatory disease and its use in the preparation of a pharmaceutical composition for the treatment of the disease, and provides a method of treatment of a subject prone to or having such a disease comprising treating the subject with the pharmaceutical composition.

MCP-1-induced chemokine receptor activation is, therefore, now shown to be involved in brain pathological events such as neurodegenerative and/or neuroinflammatory disease. A new chemokine ligand-receptor pair is thus found that contributes to an endogenous inflammatory cascade in the central nervous system which is related to the above-identified pathological conditions.

With that insight, the invention, for example, provides a method for identifying a candidate drug compound for the treatment of inflammatory or degenerative brain disease comprising testing the compound for its capacity to modulate MCP-1 binding with an orphan receptor commonly known as L-CCR in the mouse or CRAM-B in humans, in particular for the treatment of brain disease after ischemia, Alzheimer's disease or multiple sclerosis. The invention provides, for example, the characterization and the observation of mRNA expression of a novel MCP chemokine receptor CCR12 in glial cells. Evidence is here presented that astrocytes and microglia express mRNA encoding the new chemokine receptor provided here. Cloning and expressing of this new chemokine receptor revealed that MCP-1 is a chemokine ligand for this new receptor. According to the chemokine receptor nomenclature rules, we suggest to designate this receptor as “CCR12.” Since CCR12 mRNA was strongly induced by treatment with LPS, both in vitro and in vivo, further insight is provided here that this receptor plays an important role in brain immunology or brain inflammatory disease.

We thus present evidence for a new MCP-1 chemokine receptor, previously described as the orphan receptor L-CCR, when its affinity for MCP-1 was not known (27). L-CCR mRNA is expressed in mouse astrocytes and microglia and regulated by LPS both in vitro and in vivo. Since it is now found that MCP-1 is a chemokine ligand for L-CCR, we designate L-CCR as CCR12 as new chemokine receptors responsible for the well-known effects of MCP-1 on glial cells. The mRNA expression of the CC chemokine receptors CCR1-5 in cultured glial cells has been, at least partially, investigated by several groups and most studies have been performed with rat and human glial cells (Table 4). Whereas, cultured astrocytes from rat and human did not show any CCR mRNA expression; expression of CCR1 mRNA was found in mouse astrocytes (19). In rat and human microglia, mRNA expression of CCR1 (19, 20, 32) and CCR5 (18, 33, 20, 34, 32) has been reported. There are conflicting reports on the expression of CCR2 and CCR3 mRNA in cultured microglial cells. Low CCR2 mRNA expression was found in cultured microglia by Boddeke et al. (20) and McManus et al. (32), whereas no CCR2 mRNA in cultured microglia was found by others (18, 24). CCR3 mRNA expression in cultured microglia was found by He et al. (33) and McManus et al. (32), but not by Jiang et al. (18) and Boddeke et al. (20). The three reports investigating possible expression of CCR4 mRNA in glial cells failed to detect CCR4 mRNA expression (19, 20, 32). The reasons for the opposite findings concerning expression of CCR2 and 3 mRNA are currently not clear, but might be due to species variations, different culture conditions and/or different detection techniques used (Table 4).

Since very little data are available from the literature concerning CCR mRNA expression in mouse glial cells, we investigated possible mRNA expression of CCR1 to 8 and D6 in cultured mouse microglia and astrocytes using RT-PCR. The mouse chemokine receptor D6 was included in our study since it has been described as mouse CCR9 with MCP-1 binding properties (35). However, since MCP-1 signaling for D6 could not be reproduced (36), this receptor was not designated as CCR9 by the nomenclature committee and currently keeps its orphan name D6 (3). All primers used in RT-PCR experiments were positively verified using genomic mouse DNA as a template and subsequent cloning and sequencing of the PCR product. We observed mRNA expression for CCR1, 3, 5 and CCR1, 5 in cultured microglia and astrocytes, respectively, which is in good accordance with the recent literature. No other CCR mRNAs were found.

The results clearly show that, although both microglia and astrocytes respond to MCP-1 (24; own results), cultured mouse glial cells did not express CCR2 mRNA, the receptor responding to MCP-1 or D6 mRNA, a receptor which has binding properties to MCP-1. It is thus likely that cultured mouse glial cells express another receptor for MCP-1, as was already suggested by Heesen et al. (24). RT-PCR and in situ hybridization showed that both cultured astrocytes and microglia express CCR12 mRNA. The CCR12 mRNA expression in both cell types was strongly increased by stimulation with LPS. Similar results were also observed in vivo, where CCR12 mRNA expression in cortical glial cells was strongly induced by an intraperintoneal injection of LPS.

These results, therefore, clearly indicate that mouse glial cells (in vitro and in vivo) express an additional LPS-regulated chemokine receptor which has not been described in glial cells before. LPS-stimulated RAW 264.7 cells and CCR12-transfected HEK cells, which both express CCR12, responded in a concentration-dependent manner to MCP-1 in a chemotaxis assay indicating that MCP-1 is a CC chemokine ligand for CCR12. Except from MCP-1, several other chemokines (RANTES, MIP-1α, MIP-1β, MIP-3α, MCP-2, MCP-3, fractalkine, IP-10 and SLC) are known to be expressed in the brain (6-9; own observations). Among these, only RANTES, MCP-2 and MCP-3 were agonists for CCR12. The members of the MCP family, MCP-1, 2 and 3, are known to activate CCR2, but RANTES is not a chemokine ligand for CCR2, which indicates that the pharmacological profile we found for CCR12 is new and unrelated to the “ligand” profiles of other receptors (3). Taken together, our data provide the insight that the effects of MCP-1 on cultured mouse glial cells described in the literature and described here are mediated via CCR12.

Due to multiple cloning and nomination, the nomenclature of chemokine receptors has been confusing in the past (3). In order to exclude that CCR12 encodes an already known CCR paired sequence, alignment was performed. Paired sequence alignment of CCR12 with all other known mouse CC chemokine receptors (CCR1-10 and D6) revealed a percentage ID between 48% and 56% on the nucleotide level, indicating that the glial CCR12 encodes a new chemokine receptor (Table 2). This assumption is corroborated by our pharmacological findings that next to members of the MCP family, RANTES was also able to activate CCR12. Cloning of the human analogue and its expression in HEK cells revealed that MCP-1 is a chemokine ligand for the human CCR12.

Investigating the binding of biotinylated MCP-1 and MIP-1α in cultured human astrocytes, it was shown that both chemokines bind to pharmacologically different receptors since binding of MCP-1 was not competitively inhibited by MIP-1α and vice versa (37). CCR1-5 mRNA expression in human astrocytes, however, has not been found in a recent study (32). Due to the data we provide here, expression of CCR12 now offers an explanation of the pharmacological data on MCP-1 binding presented by Andjelkovic et al. (37) in human astrocytes.

Among other chemokines such as MIP-1α, RANTES, IP-10, MCP-2, MCP-3 and C10, MCP-1 is one of the most prominent chemokines in the brain. MCP-1 is induced during most types of brain injuries including Multiple Sclerosis, Alzheimer's Disease and Stroke (10-15). Within the brain glial cells (astrocytes and microglia) are predominately the cellular source of MCP-1 (13, 38, 10, 15). MCP-1 derived from glial cells is considered to be a factor controlling and/or initiating the infiltration of the damaged brain by leukocytes (17). This assumption is corroborated by a variety of findings obtained from cultured glial cells. It was found that cultured astrocytes and microglia synthesize MCP-1 upon a variety of different stimuli including LPS, IL-1β, INF-γ, TNF-α, TGF-β and β-amyloid (39-41). Moreover, MCP-1 derived from cultured astrocytes directs the migration of leukocytes across a blood-brain barrier model (16) and the secretion of metalloproteinases by cultured microglia was strongly induced by stimulation with MCP-1 (21).

Brain cells (neurons and glial cells) express various receptors for chemokines such as CCR1 and 5; CXCR2 and 4 and CX3CR (for review see 9). The expression of chemokine receptors in all intrinsic brain cells provides the insight that chemokines contribute to an endogenous inflammatory cascade in the central nervous system which is related to pathological conditions (42). Effects of chemokines on brain cells such as neuroprotection in hippocampal neurons (43), inhibition of microglial activation (44) and secretion of metalloproteinases by microglia (21) are in line with that insight. Expression of glial CCR12 mRNA in vitro and in vivo was strongly up-regulated by LPS treatment, which shows that CCR12 plays an important role in the chemokine/cytokine signaling cascade during brain inflammation.

The invention provides, among others, a method for identifying a candidate drug compound for the treatment of inflammatory or degenerative brain disease comprising testing the compound for its capacity to modulate or mimic MCP-1 binding with a chemokine receptor capable of being expressed on brain glial cells, the receptor known in the mouse as L-CCR or in humans as CRAM-B and herein also named CCR-12. Such a method is, for example, useful for finding pharmaceutical compositions for the treatment of ischemia, Alzheimer's disease or multiple sclerosis. In particular, such a method is useful when compounds are tested for their capacity to modulate or mimic MCP-1 binding, which further comprises down-regulation of the receptor, e.g., for their antagonistic characteristics. Testing can be done in vitro or in vivo, and the invention provides cells or animals provided or transfected with a recombinant nucleic acid encoding at least a functional fragment of a receptor known in the mouse as L-CCR or in humans as CRAM-B, or a functional equivalent thereof, for use in such a method according to the invention.

In a preferred embodiment, testing is provided under circumstances wherein mRNA expression of the receptor is up-regulated, such as to mimic inflammatory conditions as can be obtained after treatment with lipopolysaccharide (LPS). In the detailed description, a method according to the invention is provided wherein the capacity to modulate or mimic MCP-1 binding is measured by determining chemotaxis and/or calcium signaling; however, other ways of determining receptor-ligand binding are well known in the art and can be used as well.

It is, for example, provided to use the chemokine receptor capable of being expressed on brain glial cells, the receptor known in the mouse as L-CCR or in humans as CRAM-B, or a functional equivalent thereof, in a method according to the invention separate from cells, i.e., in a cell-free system whereby the receptor (or ligand) may be bound to a solid phase and the capacity of the candidate compound is determined by competitive assay or affinity testing.

Preferred is a use according to the invention wherein the receptor or functional equivalent thereof is expressed in a cultured cell (in vitro) to better mimic pathological conditions, especially when the cultured cell comprises a cell transfected with a nucleic acid encoding at least a functional fragment of a receptor known in the mouse as L-CCR or in humans as CRAM-B, or a functional equivalent thereof, as is, for example, shown in detail in the detailed description for an HEK cell comprising a recombinant nucleic acid encoding at least a functional fragment of a receptor known in the mouse as L-CCR or in humans as CRAM-B (herein also termed “CCR-11” or “CCR-12”) or a functional equivalent thereof. Alternatively, a transgenic mouse (e.g., a knock-in or a knock-out mouse for the nucleic acid in question) is provided for such use, especially when testing in a live animal or tissue therefrom is required.

The invention thus provides a method for obtaining or identifying an agonist or antagonist of neurodegenerative of neuroinflammatory disease comprising (if required, synthesizing and) testing a candidate agonist or antagonist compound in a method according to any one of claims 1 to 7 and determining the compound's capacity to modulate or mimic MCP-1 binding to the receptor in the method, and provides such agonists and antagonists (if required, after synthesizing the desired compound at a sufficiently large scale) for use for the preparation of a pharmaceutical composition, in particular for the treatment of neurodegenerative or neuroinflammatory disease such as Alzheimers disease, stroke, Parkinson's disease, ALS, Multiple Sclerosis, and use with other (chronic) inflammatory disease, such as atherosclerosis, arthritis, asthma (COPD) or rheuma is also foreseen, in particular to stop the progression of the above-mentioned degenerative or inflammatory diseases.

In summary, we show here evidence for the expression of a new LPS-regulated chemokine receptor in glial cells in vitro and in vivo. Also, members of the MCP family and RANTES have been identified as chemokine ligands for this receptor, and we provide the insight that the known effects of MCP-1 on mouse glial cells are mediated via CCR12-induced signaling. What is more, since expression of CCR12 mRNA is highly dependent on LPS treatment, we provide the insight that CCR12 participates in the chemokine signaling cascade during brain inflammation. The invention is further explained in the detailed description without limiting it thereto.

### BRIEF DESCRIPTION OF THE DRAWINGS

FIG. 1 illustrates RT-PCR analysis of CCR12 mRNA expression in unstimulated (C) and LPS-stimulated RAW 264.7 cells. Cells were stimulated with 100 ng/ml LPS for two hours. Number of cycles for GAPDH and CCR12 were 28 and 32, respectively. MM, molecular weight marker, highlighted band is 500 bp. Both PCR products were run in the same gel. Similar results were found in three independent experiments.

FIGS. 2A-2D depict RT-PCR analysis of chemokine receptor mRNA expression in cultured microglia (A and B) and cultured astrocytes (C and D). Experiments were carried out as described in materials and methods. A) CCR1, 3 and 5 mRNA were found in cultured microglia. B) Unstimulated microglia (C) did show basal CCR12 mRNA expression which was up-regulated by a two hour stimulation with 100 ng/ml LPS. C) In cultured astrocytes, mRNA expression for CCR1 and 5 was found. D) Control astrocytes (C) did show basal expression of CCR12 mRNA which was up-regulated by a two hour stimulation with 100 ng/ml LPS. Number of cycles for GAPDH and CCR12 were 28 and 32 respectively. MM, molecular weight marker, highlighted band is 500 bp. For B and D, both PCR products were run in the same gel. Similar results were found in three independent experiments.

FIG. 3 illustrates the effect of LPS injection on CCR12 mRNA expression in a mouse brain. RT-PCR experiments revealed that CCR12 mRNA expression in mouse brain was induced two, four and eight hours after the injection of LPS (50 μg/25 g weight). Twelve hours after the injection, CCR12 mRNA expression returned to control levels. Number of cycles for GAPDH and CCR12 were 28 and 32, respectively. MM, molecular weight marker, highlighted band is 500 bp. Both PCR products were run in the same gel. Similar results were found in three independent experiments.

FIGS. 4A and 4B show in situ hybridization in combination with immunohistochemistry showing CCR12 mRNA expression in LPS-stimulated cultured microglia and astrocytes. Cultured glial cells were stimulated for two hours with LPS (100 ng/ml) and fixed as described in materials and methods. A) Cells were incubated with ED-1 antibody to stain microglia (brown reaction product). The combination with in situ hybridization (purple reaction product) revealed that ED-1-positive microglia also express CCR12 mRNA (arrows). The ED-1-negative but CCR12 mRNA-positive cell might be an astrocyte (arrowhead). B) Cells were incubated with GFAP antibody to stain astrocytes (brown reaction product). The combination with in situ hybridization (purple reaction product) clearly showed that GFAP-positive cells also express CCR12 mRNA (arrows). The GFAP-negative but CCR12 mRNA-positive cell is most likely a microglia (arrowhead). Bar 10 μm.

FIGS. 5A-5F depict CCR12 mRNA in situ hybridization in the cortex of LPS-injected mice and identification of astrocytes as CCR12 mRNA-expressing cells. A) Lack of CCR12 mRNA expression in the control brain; only unspecific staining is visible. B) Two hours after the injection of LPS, CCR12 mRNA expression is induced in many cells. C)CCR12 mRNA returned to control levels 24 hours after the injection of LPS. D) CCR12 mRNA-positive cells in higher magnification in mouse brain two hours after LPS injection. E) Fluoresence micrograph of the same region as in D stained with anti-GFAP to detect astrocytes. F) Electronic overlay of D and E to verify that some CCR12-positive cells stain for GFAP, indicating that astrocytes are a cellular source of CCR12 mRNA. Note that there are also CCR12 mRNA-positive and GFAP-negative cells indicating that at least one other cell type different from astrocytes expresses CCR12 mRNA. Bar in A-C 50 μm; in D-F 10 μm.

FIGS. 6A and 6B show the effects of MCP-1 and RANTES on chemotaxis and intracellular calcium transients of cultured RAW 264.7 cells. A) Concentration-dependent chemotaxis of cultured RAW cells induced by MCP-1 and RANTES. The graphs show the results of a typical chemotaxis experiment performed in hexaplicate for each concentration of MCP-1 and RANTES. Data are means±SEM (n=4); similar results were obtained in four independent experiments. B) Figure shows a typical example of an induction of intracellular calcium transients in RAW cells by MCP-1 or RANTES; arrow indicates the time point of stimulation.

FIGS. 7A and 7B show the effect of MCP-1 on chemotaxis and intracellular calcium transients of CCR12-transfected HEK cells. A) Chemotaxis of MOK-transfected HEK cells was not affected by MCP-1, whereas CCR12-transfected HEK cells migrated concentration dependent when stimulated with MCP-1. The graphs show the results of a typical chemotaxis experiment performed in hexaplicate for each concentration of MCP-1. Data are means±SEM; similar results were obtained in three independent experiments. B) Typical example of a MCP-1- (100 nM) induced intracellular calcium transient in CCR 12-transfected HEK cells.

FIGS. 8A and 8B are the multiple nucleotide sequence alignment of human (hCCR12) or mouse (mcCCR12) chemokine receptor sequences herein addressed as CCR12 or CCR11.

FIG. 9 is the challenge protocol used in the COPD experiment.

FIG. 10 shows mRNA expression in the COPD experiment after one allergen provocation with 1% OVA.

FIG. 11I depicts mRNA expression in the COPD experiment after four days of repetitive allergen provocation with 1% OVA.

### DETAILED DESCRIPTION OF THE INVENTION

Materials and Methods

Chemicals

Isoflurane (Forene™) from Abbott (Baar, Switzerland); Dulbeccos modified Eagle Medium from GibcoBRL Life Technologies (Breda, Netherlands); TA vectors pCR2.1 and pCRII from Invitrogen (Leek, Netherlands); digoxigenin-conjugated UTP and alkaline phosphatase conjugated sheep-anti-digoxigenin from Boehringer Mannheim (Mannheim, Germany); recombinant mouse chemokines from Pepro Tech EC Ltd. (London, United Kingdom); antibodies for GFAP, ED-1 and MAC-1 from Chemicon (Temecula, USA); Fura-2 AM and all other chemicals from Sigma-Aldrich (Bornhem, Belgium).

Injection of LPS

For treatment with endotoxin, five week old CD-1 mice were injected intraperitoneally with LPS (50 μg/25 g weight) dissolved in sterile saline solution. Control animals received injections with 0.9% NaCl alone. At different time points after the injection, animals were decapitated under isoflurane anesthesia (five animals per time point, three for RNA preparation and two for in-situ hybridization) and brains were removed. Brains were lysed in GTC solution for RNA preparation and fixed with Zamboni's fixative by perfusion fixation for in-situ hybridization experiments.

Cell Cultures

RAW 264.7 and HEK 293 Cells

Both RAW 264.7 and HEK 293 cells were maintained in DMEM containing 10% fetal calf serum with 0.01% penicillin and 0.01% streptomycin in a humidified atmosphere (5% CO₂) at 37° C.

Mixed Astrocyte Cell Cultures and Cultured Microglia

Mixed astrocyte cell cultures were established as described previously (28). In brief, mouse cortex was dissected from newborn mouse pups (<1 d). Brain tissue was gently dissociated by trituration in phosphate-buffered saline and filtered through a cell strainer (70 μm Ø, Falcon) in DMEM. After two washing steps (200×g for 10 minutes), cells were seeded in culture dishes (Nunc, 10 cm ø) (8×10⁶ cells/dish). Cultures were maintained 6 weeks in DMEM containing 10% fetal calf serum with 0.01% penicillin and 0.01% streptomycin in a humidified atmosphere (5% CO₂) at 37° C. Culture medium was changed the second day after preparation and every six days thereafter. Microglia cultures were established as described previously (29). In brief, floating microglia were harvested from confluently mixed glial cultures and plated on new culture dishes. Microglia cultures were pure (>95%) as tested by cell-specific markers (ED-1 and Mac-1). For calcium measurements, cells were seeded on glass coverslips. For chemotaxis assays, cultured microglia were left in suspension.

Reverse Transcript Polymerase Chain Reaction (RT-PCR)

Cells and brain material were lysed in guanidinium isothiocyanate/mercaptoethanol (GTC) buffer and total RNA was extracted with slight modifications according to Chomczynski and Sacchi (30).

a) Reverse transcription: 1 μg of total RNA was transcribed into cDNA as described (28). Potential contaminations by genomic DNA were checked for by running the reactions (35 cycles) without reverse transcriptase and using GAPDH primers in subsequent PCR amplifications. Only RNA samples which showed no bands after that procedure were used for further investigation.

b) Polymerase chain reaction: 2 μl of the RT-reaction were used in subsequent PCR amplification as described (28). See table 1 for primer sequences, cycle numbers and annealing temperature. Identification of all PCR products were checked by cloning into PCR2.1 (Invitrogen) and subsequent sequencing.

Cloning and Expression of CCR12 in HEK Cells

Primers to amplify the full-length sequence for mouse CCR12 have been chosen according to the sequence for L-CCR (Accession number: AB009384). The full-length mouse CCR12 coding sequence was amplified from cDNA derived from LPS-stimulated microglia with the following primers: forward, 5′-TATCAAGCAACCTGCCTCAA (SEQ ID. NO:______); backward 5′-TGGCATAAAACAATGTGAAGAGA (SEQ ID NO:______).

Sequence similarity searches using the mouse CCR12 sequence and human databases gave high homology of mouse CCR12 with the human orphan chemokine receptor CRAM-B (Accession number: AF015525). The following primers were designed to get the full-length sequence for the human CCR12. Forward, 5′-CCCAGTGGGCAGTCTGAA (SEQ ID NO:______; backward, 5′-CTTGCATTTGGTGGATGCTA (SEQ ID NO:______.

The resulting PCR products were cloned in PCR2.1 (Invitrogen) for sequencing and subcloned into the BamHI-Not I sites of pcDNA 3.1 (Invitrogen) for transfection. 1 μg of the plasmid was transfected with 6 μl Fugene (Roche Molecular Biochemicals) in HEK 293 cells according to the manufacturer's instructions. Stably transfected cells were selected with G418 500 μg/ml for approximately two weeks and the resulting cell clones were checked by RT-PCR for CCR12 mRNA expression.

Alignment of Mouse CCR12 with Other CCRs

Paired alignment of the mouse CCR12 with other CCRs was performed using the alignment tool ClustalW at European Bioinformatics Institute (EBI), homepage http://www.ebi.ac.uk.

Determination of Intracellular Calcium

For calcium measurements, cells were cultured on poly-L-lysine coated glass coverslips. In order to load the cells with Fura-2 AM, the cells were incubated for 30 minutes at 37° C. in loading buffer containing: (in mM) NaCl 120, HEPES 5, KCL 6, CaCl₂ 2, MgCl 1, glucose 5, NaHCO₃ 22, Fura-2 AM 0.005; pH 7.4. The cover-slips were fixed in a perfusion chamber (37° C.) and mounted on an inverted microscope. Fluorometric measurements were done using a sensicam CCD camera supported by Axolab® 2.1 imaging software. Digital images of the cells were obtained at an emission wavelength of 510 nm using paired exposures to 340 and 380 nm excitation wavelength sampled at a frequency of 1 Hz. Fluorescence values representing spatial averages from a defined pixel area were recorded on-line. Increases in intracellular calcium concentrations were expressed as the 340/380 ratio of the emission wavelengths. Compounds were administered using a pipette positioned at a distance of 100-300 μm from the cells.

Chemotaxis Assay

Cell migration in response to chemokines was assessed using a 48-well chemotaxis microchamber (NeuroProbe). Chemokine stock solutions were prepared in PBS and further diluted in medium for use in the assay. Culture medium without chemokines served as a control in the assay. 27 μl of the chemoattractant solution or control medium were added to the lower wells; lower and upper wells were separated by a polyvinylpyrrolidone-free polycarbonate filter (8 μm pore size) and 50,000 cells per 50 μl were used in the assay. Determinations were done in hexaplicate. The chamber was incubated at 37° C., 5% CO₂ in a humidified atmosphere for 120 minutes. At the end of incubation, the filter was washed, fixed in methanol and stained with toluidine blue. Migrated cells were counted with a scored eyepiece (3 fields (1 mm²) per well) and migrated cells per chamber were calculated. The data are presented as mean values±S.D. and were analyzed by Students t-test. P values≦0.01 were considered significant.

Immunohistochemistry and In Situ Hybridization

Immunohistochemistry and in situ hybridization were carried out as described (31). In brief, prior to immunohistochemical processing and between the incubation steps, the sections were washed in 0.9% saline dissolved in 0.05 M Tris, pH 7.4 (TBS). All antisera were diluted in TBS containing 0.3% Triton X-100, 1% bovine serum albumin (BSA) and heparin (5 mg/ml). Sections were preincubated in 5% BSA in TBS for 30 minutes and incubated overnight with GFAP and ED-1. Antibody-antigen reactions were detected using the biotin-streptavidin method and the complex was visualized with diaminobenzidine (DAB)/H₂O₂. In the case of fluorescence detection, FITC-conjugated streptavidin was used to visualize the antibody-antigen complex. For in situ hybridization, CCR12 PCR product was cloned into the dual promoter PCR II vector and linearized. CCR12 sense and antisense probes were synthesized by run-off transcription and the use of digoxigenin-conjugated UTP according to the manufacturer's protocol (Boehringer Mannheim). Slides were rinsed in PBS and digested with 10 μg/ml proteinase K for 0.5 hour at 37° C. Subsequently, sections were rinsed in 2×SSC (1×SSC: 150 mM NaCl, 15 mM Na citrate), dehydrated in an ethanol series and dried.

Sections were hybridized overnight at 60° C. in a solution containing 50% formamide, 0.3 M NaCl, 10 mM Tris (pH 8.0), 1 mM EDTA, 0.05% tRNA, 1× Denhardt's solution and 10% dextran sulfate.

Final probe concentrations in the hybridization buffer were 1-5 ng/μl. After hybridization, the sections were treated with 10 μg/ml of ribonuclease A for 0.5 hour at 37° C. and washed in 0.1×SSC at 65° C.

The immunological detection of the digoxigenin-labeled RNA-RNA complex was preceded by a 0.5 hour pre-incubation at room temperature in 0.1 M Tris, 0.15 M NaCl, pH 7.5 (buffer 1), containing 5% BSA. Slides were incubated for two hours at room temperature with the alkaline phosphatase conjugated sheep-anti-digoxigenin, diluted 1:500 in buffer 1, containing 2% BSA. After thorough rinsing in buffer 1 and a ten minute pre-incubation in an alkaline buffer solution (ABS: 0.1 M Tris, 0.1 M NaCl, 0.05 M MgCl₂, 6H₂O, pH 9.5), the alkaline phosphatase was revealed with a freshly prepared solution of 0.34 mg/ml nitroblue tetrazoleum and 0.17 mg/ml 5-bromo-4-chloro-3 indolyl phosphate in ABS. Endogenous non-intestinal phosphatase activity was inhibited by the addition of levamisole (0.24 mg/ml) to the staining solution. The color development was done overnight and terminated by placing the slides in a buffer solution, consisting of 0.01 M Tris, 1 mM EDTA, pH 8.5. The dark purple precipitate indicating the presence of hybridized mRNA was revealed with bright-field microscopy. Control experiments included hybridization with digoxigenin-labeled sense probes and hybridization after treatment of the sections with RNAse.

Results

Expression of CCR mRNAs in RAW Cells

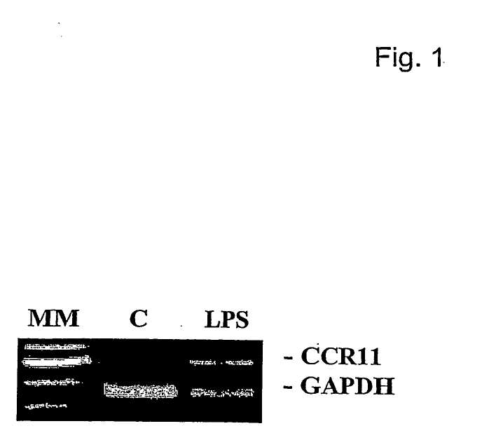

The expression of an orphan LPS-inducible chemokine receptor (L-CCR) in the mouse macrophage cell line RAW 264.7 was previously described by Shimada et al. (27). Later, we came to designate L-CCR as CCR12, and we use this term hereinafter for the sake of convenience. In order to investigate possible mRNA expression of this receptor in other cell types, we designed primers for RT-PCR experiments and validated the primers using cDNA derived from RAW 264.7 cells. Results similar to those described by Shimada et al. (1998) were found; mRNA for CCR12 was strongly up-regulated in RAW cells by stimulation with LPS (100 ng/2 hours) (FIG. 1). Using RT-PCR analysis (35 cycles), no other mRNA encoding mouse CCRs (CCR1-8 and D6) was detected in cDNA derived from control or LPS-stimulated RAW 264.7 cells. This indicates that CCR12 is the only β-chemokine receptor in these cells. Genomic mouse DNA served as a positive control for the primers (CCR1-8 and D6) used.

Expression of CCR mRNA in Cultured Mouse Astrocytes and Microglia

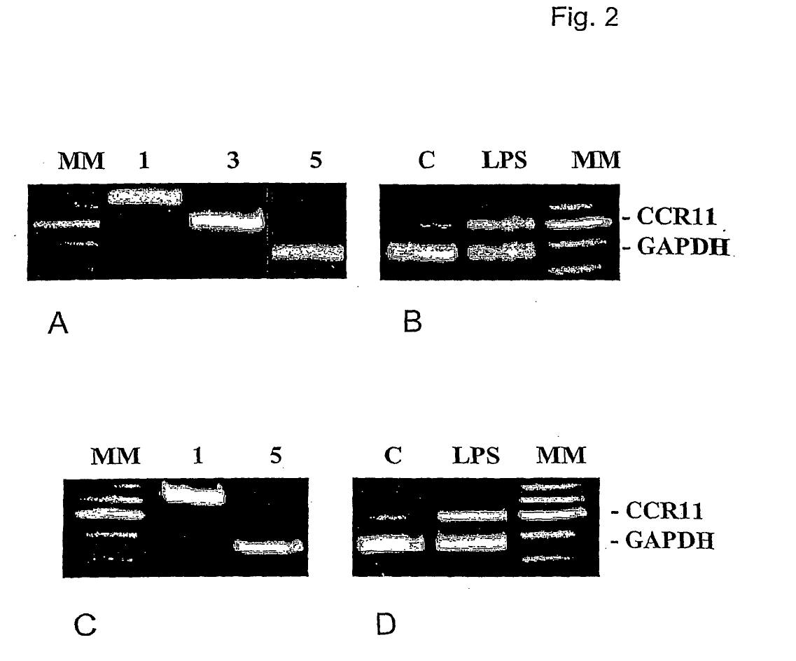

In cultured mouse microglia, mRNA for CCR1, 3 and 5 was detected (FIG. 2A). No mRNA for CCRs 2, 4, 6, 7, 8 and D6 was found in these cells (35 cycles RT-PCR) (data not shown). Untreated microglia did show basal expression levels for CCR12 mRNA and this expression was up-regulated by two hours stimulation with 100 ng/ml LPS (FIG. 2B). Similar but less pronounced effects were found after two hours stimulation with 10 and 1 ng/ml LPS (data not shown). LPS induction of CCR12 mRNA in cultured microglia peaked at two hours and declined to baseline expression after eight hours (data not shown).

Using RT-PCR, mRNA expression for CCR1 and 5 was detected in cultured astrocytes (FIG. 2C). No other CCR mRNA (2, 3, 4, 6, 7, 8 and D6) was found in these cells (35 cycles RT-PCR) (data not shown). Similar to microglia, untreated cultured astrocytes showed basal mRNA expression for CCR12, which also was up-regulated after two hours stimulation with LPS (100 ng/ml) (FIG. 2D). Treatment with 1 and 10 ng/ml LPS had a similar but less pronounced effect (data not shown). In cultured astrocytes, a comparable time dependency for the LPS effects was detected as it was found for cultured microglia (data not shown). No CCR12 mRNA expression was detected in cDNA derived from cultured cortical neurons (data not shown).

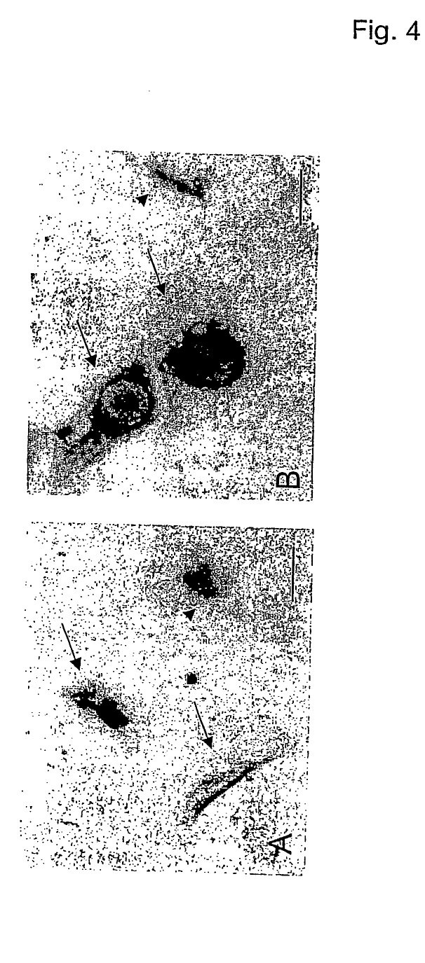

In order to verify the results obtained with RT-PCR, in situ hybridization was combined with immune histochemistry. Mixed glial cultures were stimulated for two hours with LPS (100 ng/ml) and stained with ED-1 and GFAP to detect microglia and astrocytes respectively. ED-1-positive microglia (brown reaction product) showed a positive signal for CCR12 in situ hybridization (purple reaction product) (FIG. 4A). Note that an in situ signal is also visible in ED-1-negative cells, which might be, in this case, an astrocyte (arrowhead) (FIG. 4A). CCR12-positive astrocytes (purple reaction product) (arrows) became visible by staining with GFAP (brown reaction product) (FIG. 4B). Note that also GFAP-negative cells are in situ positive, which in this case is most likely a microglial cell (arrowhead) (FIG. 4B).

Expression of CCR12 mRNA in Brain Tissue

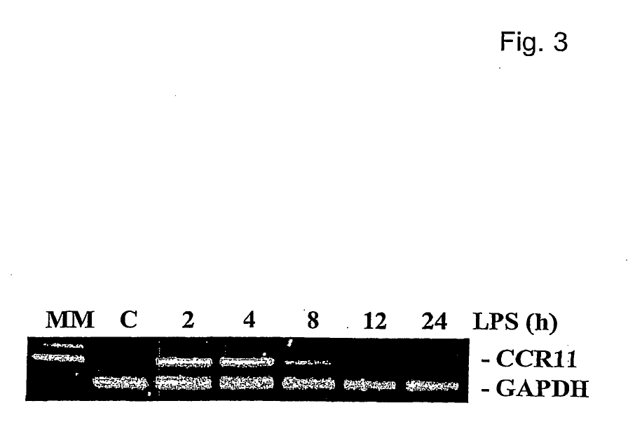

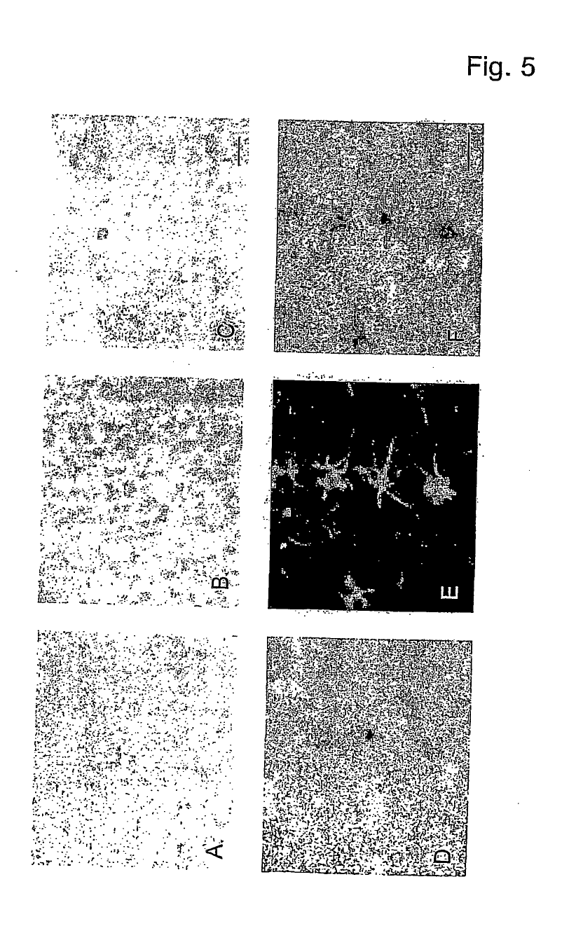

Mice were injected intraperitonally with LPS (50 μg/25 g weight) or with 0.9% NaCl and brains were removed after 2, 4, 8, 12 and 24 hours for RT-PCR analysis or in situ hybridization. Injection with a control NaCl solution did not affect expression of CCR12 mRNA in brain tissue (data not shown). In contrast, injection of LPS induced the expression of CCR11 mRNA 2, 4 and 8 hours after the injection. Twelve hours after the injection of LPS, CCR12 mRNA expression returned to baseline levels (FIG. 3). These results were verified by in situ hybridization experiments. No CCR12 mRNA-positive cells were found in control brains (FIG. 5A). Two hours after injection of LPS, many CCR12-positive cells were observed in the cortex of the LPS-treated mice (FIG. 5B). Twenty-four hours after the injection, the CCR12 in situ hybridization signal returned to control levels (FIG. 5C). Combinations of in situ hybridization (purple reaction product) (FIG. 5D) and immuno-histochemistry (GFAP fluorescence) (FIG. 5E) revealed that GFAP-positive astrocytes express CCR12 mRNA in mouse cortex (see FIG. 5F for overlay of SD and E). For technical reasons, it was not possible to colocalize CCR12 mRNA with microglial markers in vivo. Since CCR12 mRNA-positive and GFAP-negative cells were found in the brain, it is suggested that there are cell types different from astrocytes expressing CCR12 mRNA, possibly microglia as observed in cell culture studies.

Effect of MCP-1 and RANTES (CCL 5) on Chemotaxis and Calcium Signaling of RAW 264.7 Cells

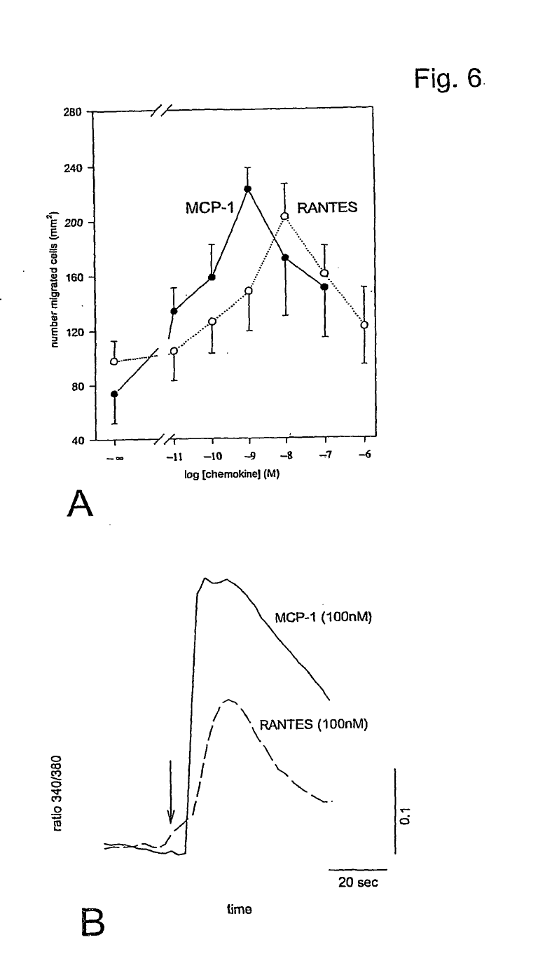

In order to find possible chemokine ligands for CCR12, chemotactic activity and mobilization of intracellular calcium were determined in LPS-treated RAW cells. MCP-1 induced concentration-dependent chemotaxis of RAW cells with an EC50 value of approximately 0.1 nM (FIG. 6A). Similar results were obtained using RANTES, which was less potent with an EC50 value of approximately 1 nM (FIG. 6A). The CC chemokine MIP-1α (CCL3) did not induce chemotaxis in RAW cells (data not shown). Both chemokines RANTES and MCP-1 were also found to induce intracellular calcium transients in RAW cells (FIG. 6B).

Chemotaxis of CCR12-Transfected HEK Cells

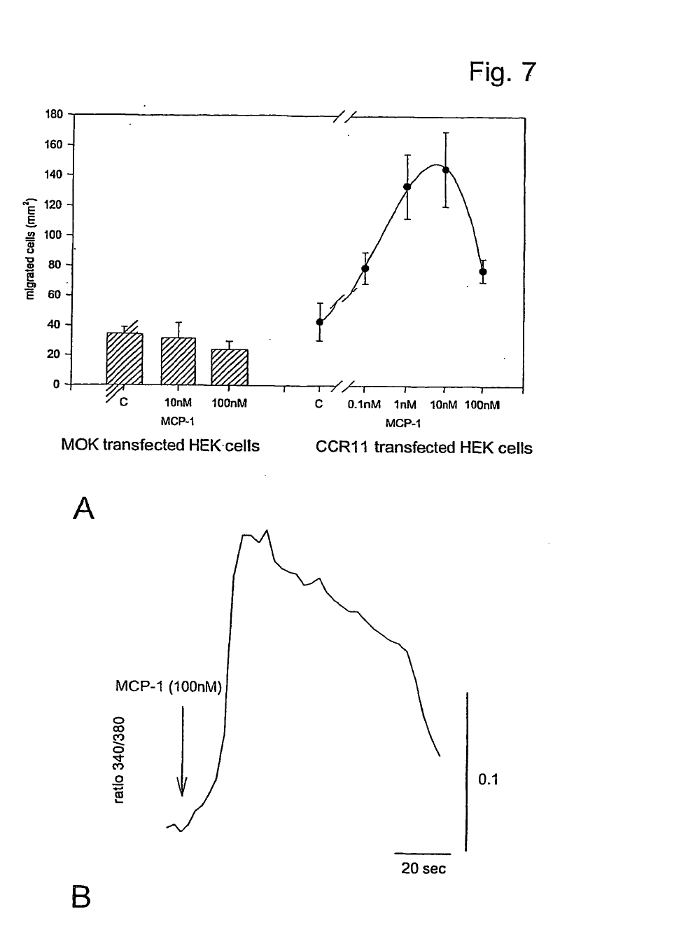

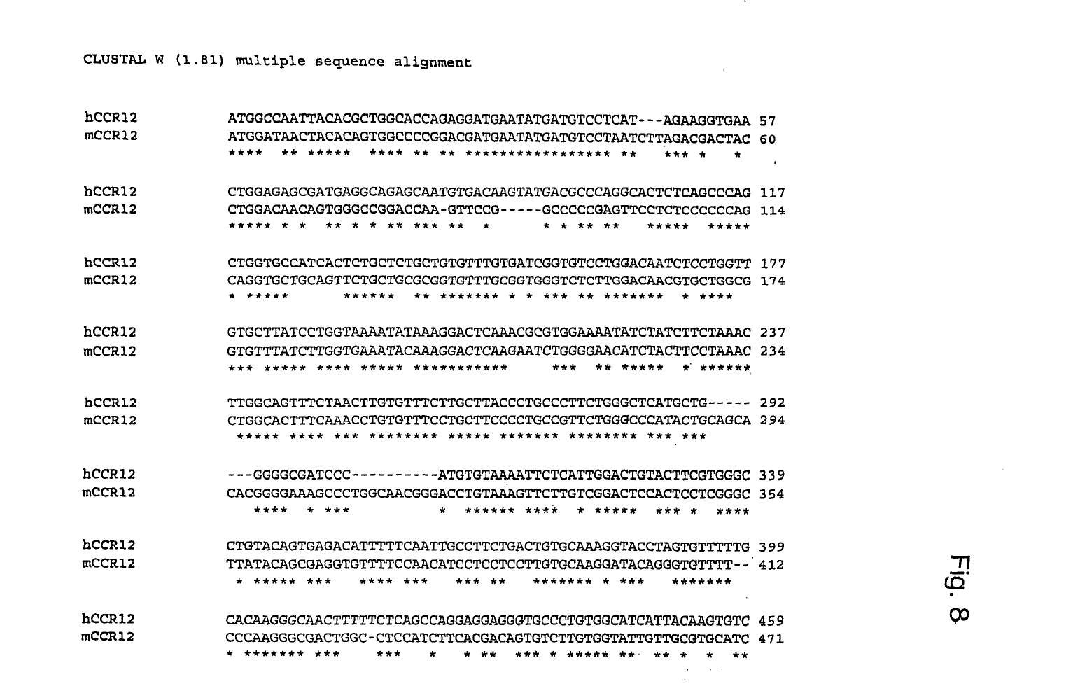

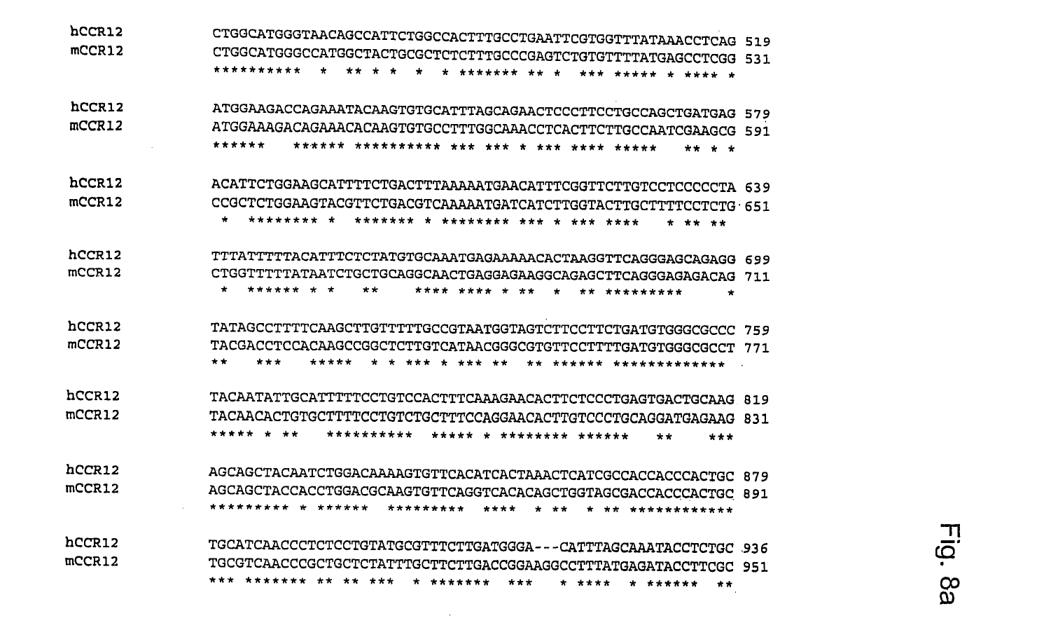

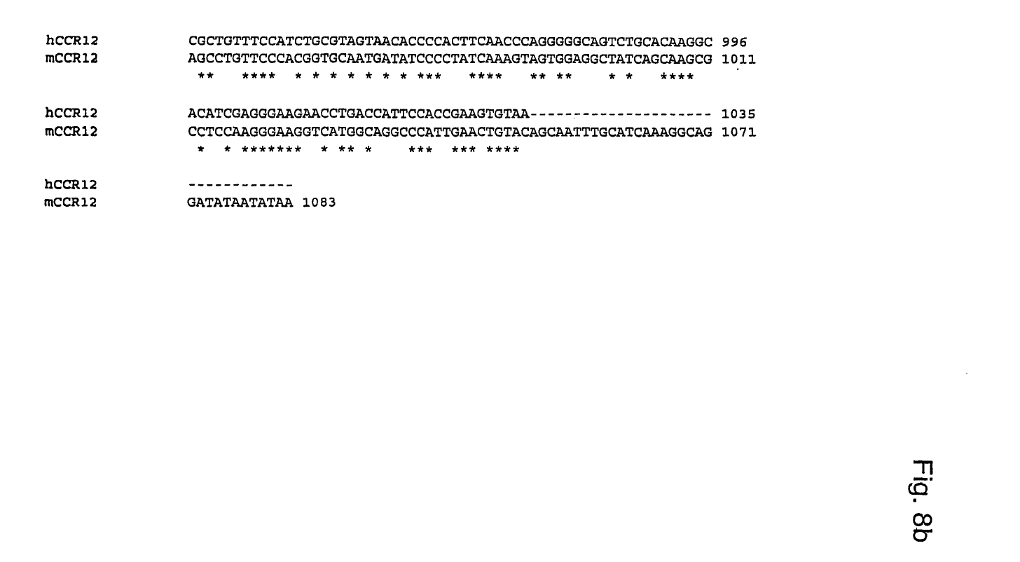

In order to further investigate its agonist responsivity, we cloned mouse CCR12 from LPS-treated microglia and subsequently the receptor was expressed in HEK 293 cells. Sequencing of the glial CCR12 revealed 99% identity with the sequence previously published for the orphan receptor (27). MOK-transfected HEK cells did not migrate towards a chemotactic gradient of MCP-1, whereas CCR12-transfected HEK cells' concentration dependently migrated in response to MCP-1 (FIG. 7A). Moreover, MCP-1 induced intracellular calcium transients in CCR12-transfected HEK cells (FIG. 7B). Among several other chemokines found in the brain (RANTES, Fractalkine (CX3CL1), MIP-1α, MIP-1β (CCL4), MIP-3α (CCL20), IP-10 (CXCL10), MCP-2 (CCL8), MCP-3 (CCL7) and SLC (CCL21)), only RANTES, MCP-2 and MCP-3 were found to induce chemotaxis of CCR12-transfected HEK cells (Table 3). In a set of preliminary experiments, we performed chemotaxis assays with HEK cells expressing the human CCR12 and verified that MCP-1 is also a chemokine ligand for human CCR12 (data not shown).

Effect of MCP-1 on Calcium and Chemotaxis of Cultured Microglia

Stimulation of chemotaxis of cultured mouse astrocytes by MCP-1 has already been shown by Heesen et al. (1996). Effects of MCP-1 on cultured microglia were only shown so far for rat microglia (20, 22) and fetal human microglia (23) but not for mouse microglia. We, therefore, determined the effects of MCP-1 on intracellular calcium transients and chemotaxis of cultured mouse microglia. Similar to microglia from other species, 10 nM MCP-1-induced chemotaxis of cultured mouse microglia: migration of untreated cells, 29±13 (cells/mm²), migration of cells stimulated with 10 nM MCP-1 170±42 (cells/mm²) (n=4). Chemotaxis was determined as described in materials and methods. Moreover, intracellular calcium transients in cultured microglia were observed upon stimulation with MCP-1 (data not shown).

TABLE 1
Primer sequences for mouse CCRs

| | | | PCR |
|---|---|---|---|
| | | | pro- |
| | | | duct |
| Gene | Primer sequences (5′-3′) | | (bp) |
| CCR1 | GTGGTGGGCAATGTCCTAGT | (SEQ ID NO:_) | 658 |
| | TCAGATTGTAGGGGGTCCAG | (SEQ ID NO:_) | |
| CCR2 | GTATCCAAGAGCTTGATGAAGGG | (SEQ ID NO:_) | 532 |
| | GTGTAATGGTGATCATCTTGTTTGGA | (SEQ ID NO:_) | |
| CCR3 | GCACCACCCTGTGAAAAAGT | (SEQ ID NO:_) | 521 |
| | CGAGGACTGCAGGAAAACTC | (SEQ ID NO:_) | |
| CCR4 | AGGCAAGGACCCTGACCTAT | (SEQ ID NO:_) | 644 |
| | GGACTGCGTGTAAGAGGAGC | (SEQ ID NO:_) | |
| CCR5 | ATTCTCCACACCCTGTTTCG | (SEQ ID NO:_) | 350 |
| | TCAGGCTTGTCTTGCTGGAA | (SEQ ID NO:_) | |
| CCR6 | GTGGTGATGACCTTTGCCTT | (SEQ ID NO:_) | 656 |
| | AGGAGGACCATGTTGTGAGG | (SEQ ID NO:_) | |
| CCR7 | AACGGGCTGGTGATACTGAC | (SEQ ID NO:_) | 596 |
| | ATGAAGACTACCACCACGGC | (SEQ ID NO:_) | |
| CCR8 | TTCCTGCCTCGATGGATTAC | (SEQ ID NO:_) | 591 |
| | GCTTCCACCTCAAAGACTGC | (SEQ ID NO:_) | |
| D6 | TCTTCATCACCTGCATGAGC | (SEQ ID NO:_) | 400 |
| | TATGGGAACCACAGCATGAA | (SEQ ID NO:_) | |
| CCR12 | CTGGCGGTGTTTATCTTGGT | (SEQ ID NO:_) | 489 |
| | AACCAGCAGAGGAAAAGCAA | (SEQ ID NO:_) | |
| GAPDH | CATCCTGCACCACCAACTGCTTAG | (SEQ ID NO:_) | 346 |
| | GCCTGCTTCACCACCTTCTTGATG | (SEQ ID NO:_) | |

TABLE 2
Comparison of CCR12 with all other cloned mouse CCRs by
nucleic acid sequence alignment

| Beta chemokine | | Percentage ID after alignment |
|---|---|---|
| receptor (mouse) | Accession Number | with glial CCR12 |
| CCR1 | U28404 | 52.8 |
| CCR2 | U51717 | 49.6 |
| CCR3 | U29677 | 54.3 |
| CCR4 | X90862 | 51.5 |
| CCR5 | U83327 | 53.1 |
| CCR6 | AB009369 | 51.6 |
| CCR7 | L31580 | 50.2 |
| CCR8 | Z98206 | 56.6 |
| CCR9 | AJ132336 | 49.1 |
| CCR10 | AF215982/AF215983 | 48.0 |
| D6 | Y12879 | 50.0 |

TABLE 3
Effect of various chemokines on chemotaxis of CCR12-transfected
HEK 293 cells

| | Chemotactic effect on CCR12-transfected HEK |
|---|---|
| Chemokine (100 nM) | cells |
| RANTES | + |
| MCP-1 | + |
| MCP-2 | + |
| MCP-3 | + |
| MIP-1^{α} | − |
| MIP-1β | − |
| MIP-3^{α} | − |
| Fractalkine | − |
| IP-10 | − |
| SLC | − |

TABLE 4
Expression profile of CCR mRNA in cultured glial cells from
human (H), mouse (M) and rat (R).

| | Astrocytes | | | Microglia | | | |
|---|---|---|---|---|---|---|---|
| | H | M | R | H | M | R | publication |
| CCR1 | | + | | | | | 19 |
| | | | | | | + | 20 |
| | − | | | + | | | 32 |
| CCR2 | | − | | | | | 24 |
| | | | | | | + | 20 |
| | | | | | | − | 18 |
| | − | | − | + | | | 32 |
| CCR3 | | | − | | | − | 18 |
| | | | | | | − | 20 |
| | | | | + | | | 33 |
| | − | | | + | | | 32 |
| CCR4 | | − | | | | | 19 |
| | | | | | | − | 20 |
| | − | | | − | | | 32 |
| CCR5 | | | − | | | + | 18 |
| | | | | | | + | 20 |
| | | | | + | | | 33 |
| | − | | | + | | | 32 |

Further Experiments

Testing in Experimental Allergic Encephalitis (EAE) in Mice

Animals

C57BL6/J mice were obtained from Jackson Laboratories and housed in groups of four, with free access to food and water. Young adult mice (8-12 weeks) were used.

Antigens

Recombinant myelin oligodendrocyte glycoprotein (MOG) (residues 35-55), obtained from S. Amor from the BPRC at TNO in Rijswijk, was used as antigen.

Induction of EAE

Immunization of the animals was done with 200 μg MOG peptide, added to PBS, emulsified by sonication for ten minutes at room temperature, in incomplete Freund's adjuvant (IFA) supplemented with 60 μg of mycobacterium. The mice were injected subcutaneously on days 0 and 7 at two sites on the back. In addition, the mice were also given i.p. 200 ng of pertussis toxin dissolved in phosphate-buffered saline (PBS). The pertussis toxin was given immediately and 24 hours later after immunization with the antigen in complete Freund's adjuvant (CFA).

Scoring in EAE

The scoring and weighing of the animals started on day eight. Grade zero meant there were no symptoms at all. At grade one the tail was paralyzed. Grade two was reached when the righting reflex was impaired. Grade three was reached when one of the hind limbs was paralyzed. Grade four was given when both hind limbs were paralyzed. Grade five is a moribund state in which all limbs are completely paralyzed. Intermediate scores of 0.5 were given. We terminated the mice at grade 3.0.

Termination of the Mice

After the termination of the mice, using Isoflurane as anesthetic, the spinal cord and the brain were taken out and frozen in liquid nitrogen for RNA analysis. For in situ hybridization, the mice were perfused with 4% paraformaldehyde (pfa) in PBS and consequently the spinal cord and brain were taken out and put in a fixative. The tissue was embedded in TissueTek, frozen and cut with a Reichert-Jung Frigocut 2800 microtome. Sections were caught on double-coated glasses.

Results

Using in situ hybridization assays, a strong increase of the CCR11 chemokine receptor was observed during EAE. This shows that the receptor is involved in MS pathology. Accordingly, the antagonists of CCR11 are of use in MS therapy.

Testing Chronic Obstructive Pulmonary Disease (Copd) in Mice Mice and In Vivo Procedures

Two weeks prior to the experiment, 8-10 week old mice C57BI/6-J were purchased from Harlan.

For sensitization (day 0), 24 mice were injected intraperitoneally with ovalbumine (OVA) 0.1 mg/mouse in Phosphor-Buffered Saline (PBS).

For induction of allergic response (day 8), 18 mice were challenged for five minutes with a 2% OVA aerosol in PBS. For control experiments, six mice were challenged for five minutes with PBS.

For allergen provocation (day 15), 18 mice were challenged for 20 minutes with a 1% OVA in PBS aerosol, six mice were challenged for 20 minutes with PBS (control). Respectively 1, 3 and 6 hours after OVA challenge and three hours after PBS challenge (control), three mice were terminated. Both lungs of all challenged mice were isolated and immediately placed in liquid Nitrogen.

On days 16, 17 and 18, mice were repetitively challenged for 20 minutes with 1% OVA (n=9) and PBS (n=3, control). On day 18, respectively 1, 3 and 6 hours after OVA challenge and three hours after PBS challenge (control), three mice were terminated. Lungs were isolated and immediately placed in liquid Nitrogen.

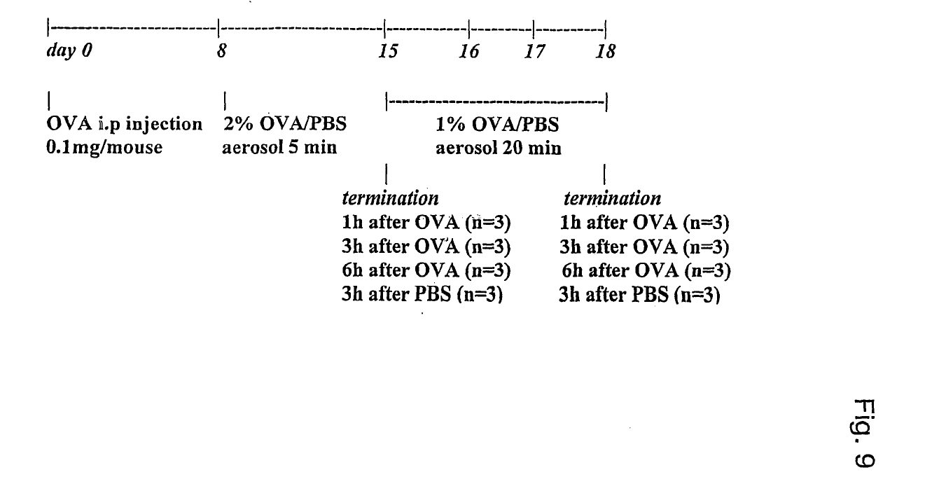

All challenge protocols were performed in a specially designed perspex cage with an internal volume of 9 L. A summary of the challenge protocol is given in FIG. 9.

RNA isolation and RT-PCR

RNA was isolated from mouse lung according to Chomczynski and Sacchi (1987) and reverse transcribed into cDNA in a final volume of 20 μl containing 1 μg RNA, 10 μl of H₂O, 1 μl of an oligo-(dT) adapter antisense primer (AP), 2 μl of 10×PCR-buffer, 2 μl of 25 mM MgCl₂, 1 μl of 10 mM dNTPs, 2 μl of 0.1 M DTT and 1 μl of Superscript II RT. After 50 minutes incubation at 42° C., the reaction was terminated by heating at 70° C. for 15 minutes. Finally, 1 μl of RNAse H was added and incubated at 37° C. for another 20 minutes.

Subsequently, 1 μl of the RT reaction was amplified by, respectively, 28 thermal cycles using specific primers for mouse GAPDH with a primer annealing temperature of 60° C., 30 thermal cycles with specific primers for mouse CCR2 (primer annealing at 56° C.), 30 thermal cycles with specific primers for mouse CCR11 (primer annealing at 56° C.) and 32 thermal cycles with specific primers for mouse MCP-1 (primer annealing at 56° C.) (FIGS. 2A, 2B). Each reaction mixture contained 1 μl of the RT reaction, 5 μl of 10×PCR-buffer (Invitek), 2.5 μl of 50 mM MgCl₂, 0.5 μl of 10 mM dNTPs (Invitek), 1 μl of each primer, 39 μl of H₂O and 0.1 μl Taq-polymerase (Invitek). The thermal cycle consisted of one minute denaturation at 94° C.; 1.5 minutes primer annealing and one minute amplification at 72° C. PCR was terminated by another seven minutes of extension at 72° C. PCR products were size fractioned on a 1.5% agarose gel.

TABLE 5
Primers used for PCR

| | | | | An- |
|---|---|---|---|---|
| | | | | neal- |
| Gene | | Primer | | ing |
| MCP-1 | Fw | GTCTCTGTCACGCTTCTGG | (SEQ ID | 56° C. |
| | | | NO:_) | |
| | Rev | GATCTCTCTCTTGAGCTTGG | (SEQ ID | |
| | | | NO:_) | |
| CCR2 | Fw | GTATCCAAGAGCTTGATGAAGGG | (SEQ ID | 56° C. |
| | | | NO:_) | |
| | Rev | GTGTAATGGTGATCATCTTGTTTGGA | (SEQ ID | |
| | | | NO:_) | |
| CCR11 | Fw | CTGGCGGTGTTTATCTTGGT | (SEQ ID | 56° C. |
| | | | NO:_) | |
| | Rev | AACCAGCAGAGGAAAAGCAA | (SEQ ID | |
| | | | NO:_) | |
| GAPDH | Fw | CATCCTGCACCACCAACTGCTTAG | (SEQ ID | 60° C. |
| | | | NO:_) | |
| | Rev | GCCTGCTTCACCACCTTCTTGATG | (SEQ ID | |
| | | | NO:_) | |

Results

Day 15, After 1 Allergen Provocation with 1% OVA

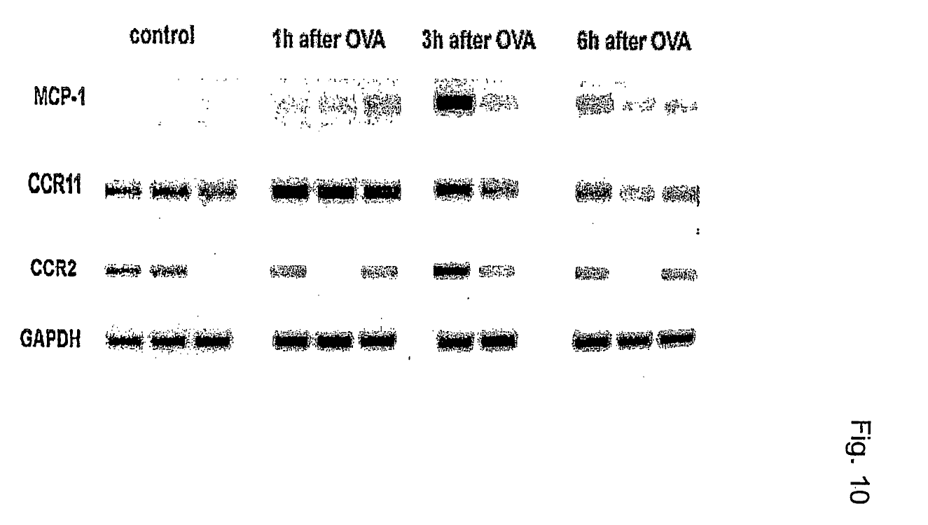

Compared to the control, MCP-1 mRNA expression is increased at respectively 1 (n—3), 3 (n=3) and 6 (n=3) hours after allergen provocation. CCR11 mRNA expression seems to be slightly increased at one hour (n=3) after allergen provocation. CCR2 mRNA expression seems to be stable. GAPDH mRNA expression was comparable in all mouse lung tissues (FIG. 10).

Day 18, After 4 Days of Repetitive Allergen Provocation with 1% OVA

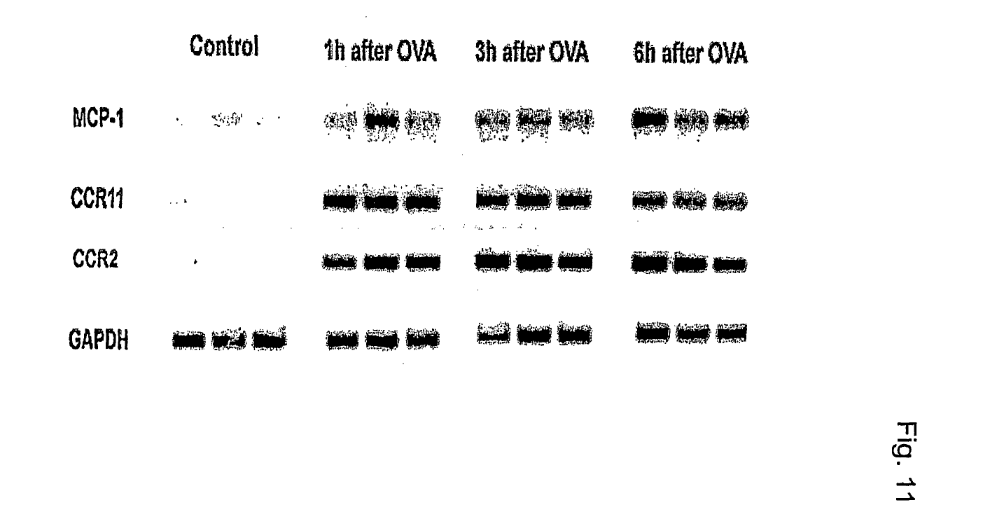

Compared to control, MCP-1, CCR11 and CCR2 mRNA expression is increased at respectively 1 (n=3), 3 (n=3) and 6 (n=3) hours after allergen challenge. GAPDH mRNA expression was comparable in all mouse lung tissues (FIG. 11).

These data show a clear involvement of CCR11 in the COPD mouse model and shows a role of CCR11 antagonists in the treatment of obstructive airway diseases such as asthma.

### REFERENCES

1. Rollins, B. J. (1997) Blood 90, 909-928.

2. Baggiolini, M. (1998) Nature 392, 565-568.

3. Murphy, P. M., M. Baggiolini, I. F. Charo, C. A. Hebert, R. Horuk, K. Matsushima, L. H. Miller, J. J. Oppenheim and C. A. Power. (2000) Pharmacol. Rev. 52, 145-176.

4. Devalaraja, M. N. and A. Richmond. (1999) Trends Pharmacol. Sci. 20, 151-156.

5. Mantovani A. (1999) Immunology Today 20, 254-257.

6. Asensio, V. C., C. Kincaid and I. L. Campbell. (1999) J. Neuro. Virol. 5, 65-75.

7. Asensio, V. C. and I. L. Campbell. (1999) Trends Neurosci. 22, 504-512.

8. Oh, J-W, L. M. Schwiebert and E. N. Benveniste. (1999) J. Neuro. Virol. 5, 82-94.

9. Glabinski, A. R. and R. M. Ransohoff. (1999) J. Neuro. Virol. 5, 3-12.

10. Gourmala, N. G., M. Buttini, S. Limonta, A. Sauter and H. W. G. M. Boddeke. (1997) J. Neuroimmunol. 74, 35-44.

11. Yamagami, S., M. Tamura, M. Hayashi, N. Endo, H. Tanabe, Y. Katsuura and K. Komoriya. (1999) J. Leukc. Biol. 65, 744-749.

12. Xia, M. Q. and B. T. Hyman. (1999) J. Neurovirol. 5, 32-41.

13. Ransohoff, R. M., T. A. Hamilton, M. Tani, M. H. Stoler, H. E. Shick, J. A. Major, M. L. Estes, D. M. Thomas and V. K. Tuohy. (1993) FASEB J. 7, 592-600.

14. Simpson, J. E., J. Newcombe, M. L. Cuzner and M. N. Woodroofe. (1998) J. Neuroimmunol. 84, 238-49.

15. van der Voom, P., J. Tekstra, R. H. Beelen, C. P. Tensen, P. van der Valk and C. J. de Groot. (1999) Am. J. Pathol. 154, 45-51.

16. Weiss, J. M., S. A. Downie, W. D. Lyman and J. W. Berman. (1998) J. Immunol. 161, 6896-6903.

17. Ransohoff, R. M. and M. Tani. (1998) Trends Neurosci. 21, 154-159.

18. Jiang, Y., M. N. Salafranca, S. Adhikari, Y. Xia, L. Feng, M. K. Sonntag, C. M. DeFiebr, N. A. Pennell, W. J. Streit and J. K. Harrison. (1998) J. Neuroimmunol. 86, 1-12.

19. Tanabe, S., M. Heesen, M. A. Berman, M. B. Fischer, I. Yoshizawa, Y. Luo and M. E. Dorf. (1997) J. Neurosci. 17, 6522-6528.

20. Boddeke, H. W. G. M., I. Meigel, S. Frentzel, N. G. Gourmala, J. K. Harrison, M. Buttini, O. Spleiss and P. Gebicke-Härter. (1999) J. Neuroimmunol. 98, 176-184.

21. Cross, A. K. and M. N. Woodroofe. (1999) GLIA 28, 183-189.

22. Cross, A. K. and M. N. Woodroofe. (1999) J. Neurosci. Res. 55, 17-23.

23. Peterson, P. K., S. Hu, J. Salak-Johnson, T. W. Molitor and C. C. Chao. (1997) J. Infect. Dis. 175,478-481.

24. Heesen, M., S. Tanabe, M. A. Berman, I. Yoshizawa, Y. Luo, R. J. Kim, T. W. Post, C. Gerard and M. E. Dorf. (1996) J. Neurosci. Res. 45, 382-391.

25. Kurihara, T. and R. Bravo. (1996) J. Biol. Chem. 271, 11603-11606.

26. Paavola, C. D., S. Hemmerich, D. Grunberger, I. Polsky, A. Bloom, R. Freedman, M. Mulkins, S. Bhakta, D. McCarley, L. Wiesent, B. Wong, K. Jarnagin and T. M. Handel. (1998) J. Biol. Chem. 273, 33157-33165.

27. Shimada, T., M. Matsumoto, Y. Tatsumi, A. Kanamaru and S. Akira. (1998) FEBS Lett. 425, 490-494.

28. Biber, K., K.-N. Klotz, M. Berger, P. J. Gebicke-Härter and D. van Calker. (1997) J. Neurosci. 17, 4956-4964.

29. Gebicke-Härter, P. J., J. Bauer, A. Schobert and H. Northoff. (1989) J. Neurosci. 9, 183-194.

30. Chomczynski, P., and N. Sacchi. (1987) Anal. Biochem. 162, 156-159.

31. Copray, J. C. V. M. and N. Brouwer. (1994) Neuroscience 63, 1125-1135.

32. McManus, C. M., K. Weidenheim, S. E. Woodman, J. Nunez, J. Hesselgesser, A. Nath and J. W. Berman. (2000) Am. J. Pathol. 156, 1441-1453.

33. He, J., Y. Chen, M. Farzan, H. Choe, A. Ohagen, S. Gartner, J. Busciglio, X. Yang, W. Hofmann, W. Newman, C. R. Mackay, J. Sodroski and D. Gabuzda. (1997) Nature 385, 645-649.

34. Spleiss, O., N. Gourmala, H. W. Boddeke, A. Sauter, B. L. Fiebich, M. Berger and P. J. Gebicke-Härter. (1998) J. Neurosci. Res. 53, 16-28.

35. Nibbs, R. J. B., S. M. Wylie, I. B. Pragnell and G. J. Graham. (1997) J. Biol. Chem. 272, 12495-12504.

36. Nibbs R. J., J. Yang, N. R. Landau, J. H. Mao and G. J. Graham. (1999) J. Biol. Chem. 274, 17478-17483.

37. Andjelkovic, A. V., D. Kerkovich, J. Shanley, L. Pulliam and J. S. Pachter. (1999) GLIA 28, 225-235.

38. Glabinski, A. R., M. Tani, R. M. Strieter, V. K. Tuohy and R. M. Ransohoff. (1997) Am. J. Pathol. 150, 617-30.

39. Hayashi, M., Y. Luo, J. Laning, R. M. Strieter and M. E. Dorf. (1995) J. Neuroimmunol. 60, 143-150.

40. Weiss, J. M. and J. W. Berman. (1998) J Neuroimmunol. 91, 190-197.

41. Johnstone, M., A. J. Geraring and K. M. Miller. (1999) J. Neuroimmunol. 93, 182-193.

42. Hesselgesser, J. and R. Horuk. (1999) J. Neuro. Virol. 5, 13-26.

43. Meucci, O., A. Fatatis, A. A. Simen, T. J. Bushell, P. W. Gray and R. J. Miller. (1998) Proc. Natl. Acad. Sci. 95, 14500-14505.

44. Zujovic, V., J. Benavides, X. Vige, C. Carter and V. Taupin. (2000) GLIA 29, 305-315.

1. A method of identifying a candidate drug compound for the treatment of an inflammatory or degenerative brain disease, said method comprising:
testing a candidate drug compound for candidate drug compound's capacity to modulate or mimic MCP-1 binding with a chemokine receptor capable of being expressed on brain glial cells, wherein said chemokine receptor is known in the mouse as L-CCR or in humans as CRAM-B.

2. The method according to claim 1 wherein said inflammatory or degenerative brain disease is selected from the group consisting of ischemia, Alzheimer's disease, multiple sclerosis, and combinations thereof.

3. The method according to claim 1 or 2 wherein the capacity to modulate or mimic MCP-1 binding comprises down-regulating the chemokine receptor.

4. The method according to claim 3 wherein the capacity is tested in vitro.

5. The method according to claim 4 wherein mRNA expression of said chemokine receptor is up-regulated.

6. The method according to claim 5 wherein the mRNA expression is up-regulated by treatment with lipopolysaccharide (LPS).

7. The method according to any one of claims 1 to 6 wherein said capacity to modulate or mimic MCP-1 binding is measured by determining chemotaxis.

8. The method according to any one of claims 1 to 8 wherein said chemokine receptor is expressed in a cultured cell.

9. The method according to claim 8 wherein said cultured cell comprises a cell transfected with a nucleic acid encoding at least a functional fragment of a receptor known in the mouse as L-CCR or in humans as CRAM-B.

10. The method according to claim 11 wherein said cell comprises a HEK cell.

11. A cell comprising a recombinant nucleic acid encoding a receptor known in the mouse as L-CCR or in humans as CRAM-B or a functional equivalent of said receptor.

12. A non-human animal comprising the cell of claim 12.

13. A process for obtaining or identifying an agonist or antagonist of degenerative of inflammatory disease, said method comprising:
testing a candidate agonist or antagonist compound in the method according to any one of claims 1 to 7, and
determining said candidate agonist or antagonist compound's capacity to modulate or mimic MCP-1 binding to said receptor in said method.

14. An agonist or antagonist of degenerative or inflammatory disease obtainable or identifiable by the method according to claim 13.

15. The agonist or antagonist of claim 14 together with a pharmaceutically acceptable excipient to form a pharmaceutical composition.

16. A method of treating a neurodegenerative of neuroinflammatory disease, said method comprising:
administering the pharmaceutical composition of claim 15 to a subject.

17. A method of identifying a candidate drug compound for the treatment of a disease selected from the group consisting of ischemia, Alzheimer's disease, multiple sclerosis, and combinations thereof, said method comprising:
testing, in vitro, a candidate drug compound for candidate drug compound's capacity to down-regulate a chemokine receptor capable of being expressed on brain glial cells, wherein said chemokine receptor is known in the mouse as L-CCR or in humans as CRAM-B.
